(19) United States
(12) Patent Application Publication
(10) Pub. No.: US 2024/0287690 A1
(43) Pub. Date: Aug. 29, 2024

(54) POROUS METAL STRUCTURE FOR PRODUCING ETHANOL FROM CARBON DIOXIDE AND METHOD FOR PRODUCING THE SAME

(71) Applicant: KOREA ADVANCED INSTITUTE OF SCIENCE AND TECHNOLOGY, Daejeon (KR)

(72) Inventors: Hye Ryung BYON, Daejeon (KR); Jiwon PARK, Daejeon (KR)

(21) Appl. No.: 18/421,443

(22) Filed: Jan. 24, 2024

(30) Foreign Application Priority Data
Feb. 27, 2023 (KR) 10-2023-0025823

(51) Int. Cl. C25B 11/053; C25B 3/07; C25B 3/26; C25B 11/031; C25B 11/091; C25D 1/08; C25D 3/38; C25D 3/46; C25D 5/10; C25D 5/18; C25D 13/22

(52) U.S. Cl. CPC C25B 11/053; C25B 3/07; C25B 3/26; C25B 11/031; C25B 11/091; C25D 1/08; C25D 3/38; C25D 3/46; C25D 5/10; C25D 5/18; C25D 13/22

(57) ABSTRACT

Provided are a porous metal structure including a substrate; a porous metal framework positioned on the substrate; and a plurality of copper (Cu) nanoclusters positioned on a surface of the porous metal framework, wherein the porous metal framework has an inverse opal structure including aligned pores, and a method for producing the same. When the porous metal structure is used as a catalyst of a carbon dioxide conversion reaction, high selectivity to ethanol may be provided.

### CROSS-REFERENCE TO RELATED APPLICATIONS

This application claims priority under 35 U.S.C. § 119 to Korean Patent Application No. 10-2023-0025823, filed on Feb. 27, 2023, in the Korean Intellectual Property Office, the disclosure of which is incorporated herein by reference in its entirety.

### TECHNICAL FIELD

The following disclosure relates to a porous metal structure allowing for the production of ethanol from carbon dioxide with high selectivity and a method for producing the same.

### BACKGROUND

In order to respond to a climate crisis caused by global warming, a study on a technology of capturing carbon dioxide and converting it into a material having a high industrial use value is being actively conducted. Carbon dioxide may be electrochemically reduced to produce a high value-added carbon compound such as carbon monoxide, methane, ethylene, and ethanol, and among them, C2 or higher compounds such as ethylene or ethanol have high utility and also are in high demand, but when carbon dioxide is reduced, selectivity to a C2 compound is low, so that it is difficult to obtain ethanol and ethylene from carbon dioxide.

When carbon dioxide is reduced, a copper (Cu)-based catalyst is mainly used for increasing selectivity to the C2 compound, and the copper (Cu) catalyst has lower selectivity to ethanol than selectivity to ethylene by a thermodynamic mechanism. Various studies for increasing the selectivity to ethanol which is industrially excellent are being conducted, but due to the low surface area and the thermodynamic mechanisms of the catalyst, it is still difficult to achieve ethanol selectivity to be desired. Therefore, a study of developing a catalyst which may improve selectivity to ethanol when electrochemically converting carbon dioxide is needed.

### SUMMARY

An embodiment of the present invention is directed to providing a porous metal structure which may significantly improve selectivity to ethanol in an electrochemical conversion process of carbon dioxide, and a method for producing the same.

In one general aspect, a porous metal structure includes: a substrate; a porous metal framework positioned on the substrate; and a plurality of copper (Cu) nanoclusters positioned on a surface of the porous metal framework, wherein the porous metal framework has an inverse opal structure including aligned pores.

In the porous metal structure according to the present invention, the pores may be interconnected to adjacent pores.

In the porous metal structure according to the present invention, the copper (Cu) nanoclusters may have an average particle diameter of 10 nm or less.

In the porous metal structure according to the present invention, the plurality of copper (Cu) nanoclusters may be derived from copper-electrodeposited thin film.

In the porous metal structure according to the present invention, the copper thin film may have a thickness of 1 to 10 nm.

In the porous metal structure according to the present invention, the porous metal framework may have a thickness of 2 to 8 μm.

In the porous metal structure according to the present invention, the pores may have an average particle diameter of 0.3 to 0.9 μm.

In the porous metal structure according to the present invention, a metal of the porous metal framework may include gold (Au), silver (Ag), zinc (Zn), or a combination thereof.

In the porous metal structure according to the present invention, the porous metal structure may have a higher peak intensity by a copper-copper bond than a peak intensity by a copper-oxygen bond in a radial distribution function obtained by Fourier transformation of an extended X-ray absorption fine structure (EXAFS) spectrum.

In another general aspect, a method for producing the porous metal structure described above is provided.

The method for producing a porous metal structure according to the present invention includes: (S10) laminating a polymer mold having an opal structure on a substrate; (S20) electrodepositing a metal in an empty space between the polymer molds; (S30) removing the polymer mold to form a porous metal framework having an inverse opal structure; and (S40) pulse-electrodepositing copper (Cu) on a surface of the porous metal framework to form a copper (Cu) thin film.

In the method for producing a porous metal structure according to the present invention, the polymer mold may have an average particle diameter of 0.3 to 0.9 μm.

In the method for producing a porous metal structure according to the present invention, in (S40), a pulse duration may be 1 to 20 ms.

In the method for producing a porous metal structure according to the present invention, in (S40), the number of pulse cycles may be 3 to 700.

In the method for producing a porous metal structure according to the present invention, (S10) may be performed by electrophoretic deposition (EPD).

In the method for producing a porous metal structure according to the present invention, the substrate may include a conductive material.

In the method for producing a porous metal structure according to the present invention, after (S10), (S11) sintering the polymer mold may be further included.

In another general aspect, a carbon dioxide conversion catalyst: includes the porous metal structure described above.

In the carbon dioxide conversion catalyst according to the present invention, the catalyst may have a selectivity to ethanol of 25% or more.

In still another general aspect, a method for producing ethanol includes: supplying carbon dioxide to a carbon dioxide conversion system including the porous metal structure described above; applying voltage to the porous metal structure to produce ethanol, and discharging the ethanol from the carbon dioxide conversion system.

Other features and aspects will be apparent from the following detailed description, the drawings, and the claims.

### BRIEF DESCRIPTION OF THE DRAWINGS

FIG. 1 is a schematic diagram illustrating a method for producing a porous metal framework according to an exemplary embodiment.

FIG. 2 is a cross-sectional SEM image of a porous metal structure according to an exemplary embodiment (a), and a high magnification SEM image of the porous metal structure (b).

FIG. 3 is high-angle annular dark-field scanning transmission electron microscope (HAADF-STEM) images of a porous metal structure according to an exemplary embodiment (a, b), and EDS mapping images of the porous metal structure (c-e).

FIG. 4 is a graph illustrating an XPS analysis spectrum of silver (a) and copper (b) according to an exemplary embodiment.

FIG. 5 is drawings illustrating an X-ray absorption spectroscopic analysis spectrum of copper (Cu) according to an exemplary embodiment, which are a k-edge X-ray absorption near edge structure (XANES) analysis spectrum (a) and an extended X-ray absorption fine structure (EXAFS) analysis spectrum (b).

FIG. 6 is a high-angle annular dark-field scanning transmission electron microscope (HAADF-STEM) image of the porous metal structure according to an exemplary embodiment (a), and an EDS mapping images of porous metal structures (b-e).

FIG. 7 is schematic diagrams illustrating a carbon dioxide reduction mechanisms depending on a copper particle size included in the porous metal structure according to an exemplary embodiment.

FIG. 8 is a graph in which potential (V vs. Cu²⁺/Cu) depending on the number of pulse cycles was measured during production of the porous metal structure according to an exemplary embodiment (a), and a graph in which Faradaic efficiency and current density were measured depending on the number of pulse cycles (b).

FIGS. 9 to 11 are graphs in which partial current density and Faradaic efficiency of a carbon dioxide conversion reaction depending on applied voltage of the porous metal structure according to an exemplary embodiment were measured.

FIGS. 12 and 13 are drawings illustrating the analysis results of Operando electrochemical shell-isolated nanoparticle-enhanced Raman spectra (SHINERS) during an electrochemical reduction process of carbon dioxide including the porous metal structure according to an exemplary embodiment as a catalyst.

FIG. 14 is a graph in which Faradaic efficiency (a) and partial current density (b) of the porous metal structure according to an exemplary embodiment were measured.

### DETAILED DESCRIPTION OF EMBODIMENTS

A porous metal structure for producing ethanol from carbon dioxide of the present invention and a method for producing the same will be described in detail. The terms used in the present specification are selected to be as common as possible and are currently widely used while considering the function of the present invention, but they may vary depending on the intention of a person skilled in the art, a convention, the emergence of new technology, or the like. The technical and scientific terms used may have, unless otherwise defined, the meaning commonly understood by those of ordinary skill in the art.

The terms such as “comprise” or “have” in the present specification and the appended claims mean that there is a characteristic or a constitutional element described in the specification, and as long as it is not particularly limited, a possibility of adding one or more other characteristics or constitutional elements is not excluded in advance.

A singular expression in the present specification and the appended claims includes a plural expression, unless otherwise explicitly specified as singular. In addition, a plural expression includes a singular expression, unless otherwise explicitly specified as plural.

In addition, the numerical range used in the present specification includes all values within the range including the lower limit and the upper limit, increments logically derived in a form and span of a defined range, all double limited values, and all possible combinations of the upper limit and the lower limit in the numerical range defined in different forms. Unless otherwise defined in the specification of the present invention, values which may be outside a numerical range due to experimental error or rounding off of a value are also included in the defined numerical range.

The term of degree “about” and the like used in the present specification and the attached claims are used in the sense of covering an allowable error when the allowable error exists.

A “C2 compound” in the present specification refers to a compound having two carbons, and may include ethanol and ethylene.

An “asterisk symbol (*)” in the present specification refers to a material adsorbed on a surface. As an example, *CO refers to carbon monoxide adsorbed on a surface.

“Selectivity” in the present specification refers to Faradaic efficiency which shows a ratio of charge quantity used to produce a product to total charge quantity, in a carbon dioxide conversion reaction.

In order to produce ethanol having high industrial value by capturing carbon dioxide which is the main component of greenhouse gas, copper (Cu) has been used as a catalyst, but a copper catalyst has low selectivity to ethanol as compared with ethylene by thermodynamic mechanism. In order to increase ethanol conversion efficiency, in the recent study, gold, silver, or zinc is used together with the copper catalyst, thereby improving selectivity to ethanol. However, it is still difficult to achieve ethanol selectivity to be desired due to a low surface area.

Thus, after in-depth study, the present applicant developed a carbon dioxide conversion catalyst having improved selectivity to ethanol by using a porous metal structure having a maximized surface area of a catalyst.

In one general aspect, a porous metal structure includes: a substrate; a porous metal framework positioned on the substrate; and a plurality of copper (Cu) nanoclusters positioned on a surface of the porous metal framework, wherein the porous metal framework has an inverse opal structure including aligned pores.

Since the three-dimensional porous metal framework adopts an inverse opal structure including aligned pores to improve a surface area, active sites of the catalyst are increased to improve reaction efficiency. In addition, a plurality of copper (Cu) nanoclusters having high reactivity may be positioned on the surface of the porous metal framework to maximize a specific surface area. In particular, when the porous metal structure is used as a catalyst of a carbon dioxide conversion process, selectivity to ethanol may be increased, which is thus favorable.

A nanocluster refers to a particle which has a particle size of several nanometers or less and is thermodynamically metastable. Nanoclusters having a significantly small size as compared with general nanoparticles having an average particle size of tens to hundreds of nanometers have a maximized particle specific surface area, and also have intermediate properties between atoms and nanoparticles to have physical and chemical properties different from nanoparticles. In particular, when the nanoclusters are used as a catalyst, reactivity is improved due to the properties different from the nanoparticles, thereby providing excellent efficiency and selectivity.

In an exemplary embodiment, copper (Cu) nanoclusters may have an average particle diameter of 10 nm or less or 8 nm or less, preferably 6 nm or less, and unlimitedly, 30 nm or more, 50 nm or more, or 100 nm or more. Nanoclusters having properties different from general nanoparticles with their significantly small particle diameter may be used to improve a specific surface area. In addition, since a porous metal framework is partly exposed to a catalyst surface as well as copper nanoclusters, an environment prone to forming carbon monoxide which is a reaction intermediate from carbon dioxide may be created.

When copper nanoparticles having an average particle diameter of tens to hundreds of nanometers rather than copper nanoclusters are included on the porous metal framework, the specific surface area of the porous metal framework is narrowed to decrease a carbon monoxide conversion rate, and a particle size is increased to limit space when a reactant and a reaction intermediate positioned on the surface of the porous metal structure move, thereby causing a concern about reducing ethanol production efficiency. Therefore, the porous metal structure includes copper nanoclusters having a significantly small average particle diameter, thereby improving selectivity to ethanol, which is thus favorable.

In an exemplary embodiment, the plurality of copper (Cu) nanoclusters may be derived from a copper (Cu)-electrodeposited thin film. Specifically, a copper (Cu) thin film which is electrodeposited on the surface of the porous metal framework may form copper nanoclusters during a carbon dioxide reduction reaction process. The copper thin film may have a thickness of 1 to 10 nm or 2 to 8 nm, preferably 3 to 7 nm, and unlimitedly 30 nm or more, 50 nm or more, or 100 nm or more.

Since copper nanoclusters are formed from the copper thin film having the thickness, the porous metal framework is exposed to the surface during a catalyst reaction to facilitate contact with carbon dioxide, and thus, a large amount of carbon monoxide may be produced. The produced carbon monoxide may come into contact with copper nanoclusters to be converted into ethanol. Since there is no spatial constraint by copper nanoclusters having a very small particle diameter, ethanol may be easily formed without carbon monoxide interference.

In a specific example, the porous metal structure may have a higher peak intensity by a copper-copper bond than a peak intensity by a copper-oxygen bond in a radial distribution function obtained by Fourier transformation of an extended X-ray absorption fine structure (EXAFS) spectrum. When the surface of the copper nanoclusters is partially naturally oxidized, there is a concern that selectivity to ethylene rather than selectivity to ethanol, is increased. Therefore, a copper-copper bond is dominantly included as compared with a copper-oxygen bond, so that carbon monoxide is adsorbed on the surface of unoxidized copper nanoclusters, thereby converting carbon dioxide into ethanol more efficiently. In addition, without being necessarily bound to the interpretation, in copper nanoclusters on which naturally oxidized copper is dominantly positioned, oxygen positioned on the surface reacts with carbon monoxide which is a reaction intermediate, thereby oxidizing carbon monoxide to reduce ethanol selectivity.

In an exemplary embodiment, it is preferred that the metal of the porous metal framework includes a carbon dioxide reduction catalyst having high carbon monoxide selectivity, and as an example, the metal may include one selected from the group consisting of gold (Au), silver (Ag), zinc (Zn), or a combination thereof, and preferably silver (Ag).

The porous metal framework includes a metal catalyst having high carbon monoxide selectivity, thereby producing carbon monoxide which is a reaction intermediate in a large amount during production of ethanol from carbon dioxide. Carbon dioxide is converted into carbon monoxide by the porous metal framework and adsorbed on the surface, and subsequently, a pathway in which copper nanoclusters positioned on the surface of the porous metal framework and carbon monoxide come into contact to produce ethanol is provided, thereby providing high ethanol conversion efficiency as compared with a single copper catalyst.

In order to provide a pathway in which a reactant to reach the surface of the porous metal structure may react with the porous metal framework, and then move well to copper nanoclusters, it is preferred that pores included in the porous metal framework are interconnected to adjacent pores. As an example, the pores may have an average particle diameter of 0.3 to 0.9 μm or 0.4 to 0.8 μm, and preferably 0.5 to 0.7 μm. A space in which the reactant is adsorbed on the surface of the porous metal structure and rapidly performs a reaction, and a product may be easily desorbed from the surface of the porous metal structure is provided through the pores having the average particle diameter in the range, thereby preventing decline in carbon dioxide conversion efficiency by a plugging phenomenon in which the reactant or product is precipitated in pores to block the pores.

In an exemplary embodiment, the porous metal framework may have a thickness of 2 to 8 μm or 3 to 7 μm, preferably 4 to 6 μm. The plurality of aligned pores may be laminated to improve the surface area of the porous metal structure.

The present invention includes a method for producing the porous metal structure described above. In the description of the method for producing a porous metal structure, since the material, the structure, the shape, the size, and the like of the porous metal structure are the same as or similar to those of the porous metal structure described above, the method for producing a porous metal structure according to the present invention includes the descriptions described above in the porous metal structure.

The method for producing a porous metal structure according to the present invention includes: (S10) laminating a polymer mold having an opal structure on a substrate; (S20) electrodepositing a metal in an empty space between the polymer molds; (S30) removing the polymer mold to form a porous metal framework having an inverse opal structure; and (S40) pulse-electrodepositing copper (Cu) on a surface of the porous metal framework to form a copper (Cu) thin film.

In (S10), a polymer mold may be densely laminated in a hexagonal structure on a substrate by electrophoretic deposition (EPD). Specifically, the polymer mold may be laminated on the substrate by immersing the substrate in a solution in which the polymer mold is dispersed and applying voltage. In order to use the substrate as a working electrode, the substrate may be conductive. As a specific example, the substrate may include a conductive metal such as gold (Au) or a carbon-based material such as glassy carbon to impart conductivity to a substrate, and the substrate may include a gas diffusion layer, but the present invention is not limited to a specific material of the substrate.

The polymer mold may include polystyrene (PS), polymethyl methacrylate (PMMA), polyamide (PA), polyvinylidene fluoride (PVDF), polyethylene oxide (PEO), polyphenylene oxide (PPO), and the like, and preferably polystyrene (PS). By controlling the particle diameter of the polymer mold, the size of pores included in the porous metal framework produced may be adjusted. Specifically, the polymer mold may have an average particle diameter of 0.3 to 0.9 μm or 0.4 to 0.8 μm, preferably 0.5 to 0.7 μm.

After (S10), (S11) sintering the polymer mold may be further included. The substrate including the polymer mold may be sintered to strengthen a binding force between the substrate and the polymer mold and between the polymer molds. It is preferred that a sintering temperature is near a glass transition temperature (Tg) of the polymer mold, specifically between the glass transition temperature and a melting temperature of a polymer, in terms of strengthening a binding force.

After laminating the polymer mold on the substrate, (S20) electrodepositing a metal in an empty space between the polymer molds may be performed. The substrate including the polymer mold is used as a working electrode and a metal is used as a counter electrode, the working electrode and the counter electrode are immersed in a water-based electrodeposition solution including metal ions, and voltage may be applied to electrodeposit metal. Before immersing the working electrode in the water-based electrodeposition solution, the working electrode may be first immersed in the polar organic solvent to prevent metal from being unevenly electrodeposited on a hydrophobic polymer mold or the polymer mold from being released from the substrate. As an example, the polar organic solvent may include ethanol, but the present invention is not limited thereto.

After the metal is electrodeposited in an empty space of the polymer mold, (S30) removing the polymer mold to form a porous metal framework having an inverse opal structure may be performed. The substrate including the polymer mold is immersed in the organic solvent which may dissolve the polymer mold, thereby dissolving the polymer mold. In a specific example, when the polymer mold includes polystyrene (PS), toluene is used as the organic solvent to dissolve the polymer mold, but the present invention is not limited thereto, and any organic solvent which may dissolve the polymer mold may be used. The porous metal framework including aligned pores having a hexagonal structure may be produced by the above method.

Thereafter, pulse-electrodepositing copper (Cu) on the surface of the porous metal framework having an inverse opal structure to form a copper (Cu) thin film may be performed to produce a porous metal structure. In pulse electrodeposition, a porous metal framework as a working electrode and a copper (Cu) foil as a counter electrode are immersed in a water-based electrodeposition solution containing a copper ion, and then pulse voltage may be applied to deposit a copper thin film on the porous metal structure. Copper of an ultrathin film may be isotropically deposited even on the surface of a three-dimensional porous metal framework including a plurality of pores, by pulse electrodeposition. The working electrode may be immersed in a polar organic solvent before being immersed in the water-based electrodeposition solution as in (S20), thereby preventing non-uniform electrodeposition of the copper thin film due to the hydrophobicity of the porous metal framework.

The pulse electrodeposition is favorable, since the content and the thickness of copper which is electrodeposited on the surface of the porous metal framework may be precisely controlled. Specifically, a pulse duration may be 1 to 20 ms, 3 to 17 ms, or 5 to 15 ms, preferably 8 to 12 ms. After a pulse is applied during the time, a pause time of 2 to 7 seconds or 3 to 6 seconds, preferably 4 to 5 seconds may be given. A pulse of high current density may be applied for a short time to produce a large amount of small copper particle nuclei, and copper ions in the electrodeposition solution are depleted due to pulse electrodeposition to prevent dendritic copper from being produced. The consumed copper ions on the electrode surface may be supplemented by taking a long pause time as compared with the pulse duration. When performing one pulse time and pause time is referred to as a pulse cycle, the pulse cycles may be performed 3 to 700 times, 5 to 600 times, or 12 to 500 times to electrodeposit the copper thin film having a desired thickness.

In an example, the porous metal structure having an inverse opal structure described above may be a carbon dioxide conversion catalyst, and more specifically, the porous metal structure may be a catalyst for producing ethanol.

Conventionally, a copper catalyst used for producing a C2 compound by reducing carbon dioxide has high selectivity to ethylene, but low selectivity to ethanol. Therefore, in order to improve selectivity to ethanol of the C2 compounds, a porous metal structure having an inverse opal structure is included as a catalyst to form a carbon monoxide intermediate, and then a reaction pathway to produce ethanol by dimerization of carbon monoxide is derived, thereby effectively improving selectivity to ethanol.

As described above, in order to produce carbon monoxide which is a reaction intermediate from carbon dioxide, copper and a metal catalyst having high carbon monoxide selectivity may be used together as a carbon dioxide conversion catalyst. As the metal, a catalyst which is known to have high carbon monoxide selectivity such as gold (Au), silver (Ag), and zinc (Zn) may be included, and preferably silver (Ag) may be included. Only when a large amount of carbon monoxide which is a reaction intermediate is adsorbed on the surface of the catalyst, ethanol selectivity due to dimerization of carbon monoxide on the surface of copper may be increased, and thus, selectivity to carbon monoxide which is a reaction intermediate may be improved by a porous metal structure having a large specific surface area.

In an exemplary embodiment, since copper nanoclusters having a very small particle size are included, the surface of a porous metal framework is exposed, and carbon dioxide infiltrating the pores of the catalyst may be converted into carbon monoxide on the surface of the porous metal framework. The converted carbon monoxide may move to the surface of the copper nanoclusters on the surface of the porous metal framework to produce ethanol by dimerization of carbon monoxide. It is favorable to improve the surface area of the catalyst, since the amount of the product may be significantly increased as compared with a flat plate-shaped silver-copper composite catalyst.

In a specific example, the carbon dioxide conversion catalyst may have the ethanol selectivity of 25% or more, 28% or more, or 30% or more, preferably 33% or more, and unlimitedly 50% or less. Production of ethanol by the mechanism may be implemented by a carbon dioxide conversion system including an electrochemical cell provided with an oxidation electrode, a reduction electrode, and an ion exchange membrane.

The method for producing ethanol according to the present invention includes: supplying carbon dioxide to a carbon dioxide conversion system including the porous metal structure described above; applying voltage to the porous metal structure to produce ethanol, and discharging the ethanol from the carbon dioxide conversion system.

In an exemplary embodiment, the carbon dioxide conversion system may include a sandwich type electrochemical cell. The sandwich type electrochemical cell may have a structure in which a reduction electrode current collector, a reduction electrode, an ion exchange membrane, an oxidation electrode, and an oxidation electrode current collector are sequentially laminated, but the present invention is not limited to the specific type of the electrochemical cell.

The reduction electrode may include the porous metal structure described above as a catalyst, and an ion exchange membrane may be installed between the reduction electrode and the oxidation electrode to prevent a crossover phenomenon in which ions produced in each electrode move to a counter electrode. Each current collector may include carbon dioxide supply flow path and discharge flow path and a production discharge flow path. Carbon dioxide is bubbled in an electrolyte solution by the carbon dioxide supply flow path to continuously supply a reactant, and then voltage is applied to perform a carbon dioxide conversion reaction. A voltage of −1.15 to −0.9 V or −1.13 to −0.95 V as compared with a reversible hydrogen electrode may be applied, preferably when a voltage of −1.1 to −1.0 V as compared with a reversible hydrogen electrode is applied, ethylene production efficiency may be increased, which is thus favorable.

As described above, a carbon dioxide conversion system using the porous metal structure as a catalyst is used to efficiently consume carbon dioxide, which is converted into ethanol having a high industrial use value, thereby providing an environmentally friendly process according to greenhouse gas reduction.

Hereinafter, the present invention will be described in detail by the examples.

### Example 1

### Lamination of Polymer Template

A silicon (Si) wafer was coated with titanium (Ti) having a thickness of 10 nm and gold (Au) having a thickness of 200 nm sequentially by electron beam deposition. A wafer having a size of 2.5×2 cm² was ultrasonically washed (18.2 Mω-cm) for 10 minutes in acetone (purity: 99.5%), isopropyl alcohol (HPLC grade), anhydrous ethanol (99.9%), and ultrapure water (DI water) to prepare a substrate.

Spherical polystyrene beads (Thermo Fisher Scientific) including a sulfonate group having an average particle diameter of 600 nm were washed in ultrapure water and ethanol. The cleaning was performed by repeating centrifugation and redispersion. The washed polystyrene beads were dispersed in a solution in which 93.75% of ethanol, 5% of deionized water, and 1.25% of ammonia water (NH₄OH) having a concentration of 30 wt % were mixed, and the pH of the solution was adjusted to 10.

The polystyrene beads were laminated on the substrate by electrophoretic deposition (EPD). A two-electrode cell using a titanium (Ti) mesh electrode as a counter electrode and a substrate as a working electrode was immersed in a solution in which polystyrene beads were dispersed, and then was deposited for 10 minutes at a constant current density of 20 μA/cm². Thereafter, the substrate on which the polystyrene beads were deposited was taken out, and sintered at 100° C. for 1 hour.

### Production of Porous Metal Framework

A potassium solution in which 1.6 g of Potassium metabisulfite (K₂S₂O₅) was dissolved in 10 mL of deionized water was added to a silver solution in which 1.6 g of silver nitrate (AgNO₃, reagent plus, Aldrich) was mixed with 10 mL of deionized water and mixed, and the mixture was dropped into a sodium solution in which 8 g of sodium hyposulphite (Na₂S₂O₃, Bioultra, Aldrich) and 15 mL of deionized water were mixed to prepare a mixed solution. Thereafter, 1 g of ammonium acetate (CH₃COONH₄, ≥99.99%, Aldrich) and 84 mg of thiosemicarbazide (H₂NCSNHNH₂, 98%, Aldrich) were sequentially introduced to the mixed solution, and 0.742 mL of acetic acid (ACS reagent, Aldrich) was added to adjust pH, thereby preparing a water-based electrodeposition solution including silver ions.

Thereafter, a substrate on which polystyrene beads were deposited was used as a working electrode, and the working electrode was immersed in ethanol, and then in a water-based electrodeposition solution. A silver (Ag) foil as a counter electrode was also immersed in the water-based electrodeposition solution, and a constant current density of 5 mA/cm² was applied for 6 minutes to electrodeposit silver (Ag) in an empty space between polystyrene beads. After silver was electrodeposited, it was immersed in toluene overnight to dissolve polystyrene beads, and toluene and acetone were used to perform ultrasonic washing for 5 minutes, respectively, to remove residual polystyrene, thereby producing a porous metal framework having an inverse opal structure. The porous metal framework had a thickness of 4.5 μm.

### Production of Porous Metal Structure

0.1 M of copper sulfate pentahydrate (CuSO₄·₅H₂O, 99.999%, Aldrich) and 0.2 M of sulfuric acid (ACS reagent, Aldrich) were mixed to prepare a water-based electrodeposition solution based on copper sulfate under an acid condition. A copper foil as a counter electrode and a porous metal framework as a working electrode were immersed in the water-based electrodeposition solution based on copper sulfate to perform pulse electrodeposition. At this time, the porous metal framework was first immersed in ethanol before being immersed in the water-based electrodeposition solution based on copper sulfate. Applying a pulse for 10 ms at a current density of −100 mA/cm² and pausing for 4.99 seconds were set as one cycle, and 240 cycles were repeated to electrodeposit a copper thin film having a thickness of 6 nm, thereby producing a porous metal structure.

### Example 2

A porous metal structure was produced in the same manner as in Example 1, except that pulse cycles were repeated three times when producing the porous metal structure.

### Example 3

A porous metal structure was produced in the same manner as in Example 1, except that pulse cycles were repeated 12 times when producing the porous metal structure.

### Example 4

A porous metal structure was produced in the same manner as in Example 1, except that pulse cycles were repeated 1200 times when producing the porous metal structure.

### Example 5

A porous metal structure was produced in the same manner as in Example 1, except that a porous metal framework having a thickness of 1.5 μm was produced and pulse cycles were repeated 10 times when producing the porous metal structure.

### Example 6

A porous metal structure was produced in the same manner as in Example 5, except that pulse cycles were repeated 80 times when producing the porous metal structure.

### Comparative Example 1

A flat plate-shaped silver-copper composite thin film was produced by sequentially electrodepositing silver (Ag) and copper (Cu) on the substrate in the same manner as in Example 1, except that the polymer mold was not laminated on the substrate.

### Comparative Example 2

A flat plate-shaped copper thin film was produced by coating a silicon wafer with titanium (Ti) having a thickness of 100 nm and copper having a thickness of 1 μm sequentially by electron beam deposition.

### <Measuring Equipment>

SEM image: It was observed with a high resolution field emission scanning electron microscope (SU8230, Hitachi).

HAADF-STEM image and EDS mapping image: A catalyst before performing a carbon dioxide conversion reaction was observed using Titan Themis Z (Thermo Fisher Scientific), and a catalyst after performing the carbon dioxide conversion reaction was observed using Titan cubed G2 60-300 (Thermo Fisher Scientific).

XPS analysis: Al Kα source, K-alpha (Thermo VG Scientific) was used to perform X-ray photoelectron spectroscopy.

XANES and EXAFS: In a 8C Nano-XAFS beam line from Pohang Accelerator Laboratory (PAL), an X-ray absorption near-edge structure (XANES) spectrum and a k²-loaded Fourier transformed extended X-ray absorption fine structure (EXAFS) spectrum was obtained to observe a short-distance order structure of a sample. The obtained Cu K-edge spectrum was analyzed by an Athena software of a Demeter package with an IFEFFIT library.

SHINERS: A Raman microscope (Andor Shamrock SR303i, Oxford Instruments) using a 633 nm excitation laser source (output: 65 mW, 50% ND filter) was used. A water immersion objective lens (LUMPLFLN40XW, Olympus, numerical aperture: 0.8) was covered with a PFA film (thickness: 0.05 mm, AS ONE) and used in an electrochemical cell. A drop of DI water was injected between the PFA film and the objective lens to remove residual air to match a refractive index, and then each potential applied before collecting the Operando electrochemical SHINER was maintained for at least 5 minutes (SP-150, Biologic), and a steady state condition was maintained. Thereafter, an Operando electrochemical SHINERS graph was collected by an acquisition time of 1 sec and 30 repetitions.

### Experimental Example 1: Evaluation of Carbon Dioxide Electrochemical Conversion Performance

The porous metal structures produced by the methods of Examples 1 to 4 and Comparative Examples 1 and 2 were used as a catalyst to perform a carbon dioxide conversion reaction. A carbon dioxide conversion reaction was performed in a sealed sandwich type electrochemical cell. A dimensionally stable anode (Innochemtech) including the porous metal structure as a catalyst on one surface was used as a working electrode and a double junction silver/silver chloride (Ag/AgCl) electrode saturated with potassium chloride was used as a counter electrode and a reference electrode, respectively. An anion exchange membrane (Selemion AMV-n, AGC Engineering Co. Ltd.) was interposed between the working electrode and the counter electrode. An electrolyte solution was prepared by dissolving a 0.2 M potassium hydrogen carbonate (K₂CO₃, 99.995%, Aldrich) aqueous solution in deionized water and then bubbling high purity carbon dioxide gas (99.999%, Deokyang) overnight.

The pH of the potassium hydrogen carbonate electrolyte solution saturated with carbon dioxide was 6.8, and 6 mL of the electrolyte solution was taken and supplied to an electrochemical cell. In the electrochemical cell, carbon dioxide gas was continuously bubbled at a flow rate of 20 sccm to the working electrode and the counter electrode. Each electrode was electrically connected, and a current of 30 mA/cm² was applied to perform a carbon dioxide conversion reaction for 3 hours. The flow rate to the working electrode and the counter electrode was controlled by a mass flow controller (Linetech and KOFLOC), and the flow rate of a product gas was monitored by a mass flow meter (Linetech).

FIG. 1 is a schematic diagram illustrating a method for producing a porous metal framework according to an exemplary embodiment. A polymer mold was laminated on a substrate, a metal was electrodeposited on an empty space between the polymer molds, and then the polymer mold was removed to produce a porous metal framework including pores. The polymer mold was laminated by electrophoretic deposition (EPD) to produce the porous metal framework, of which the pores may be densely aligned in a hexagonal structure.

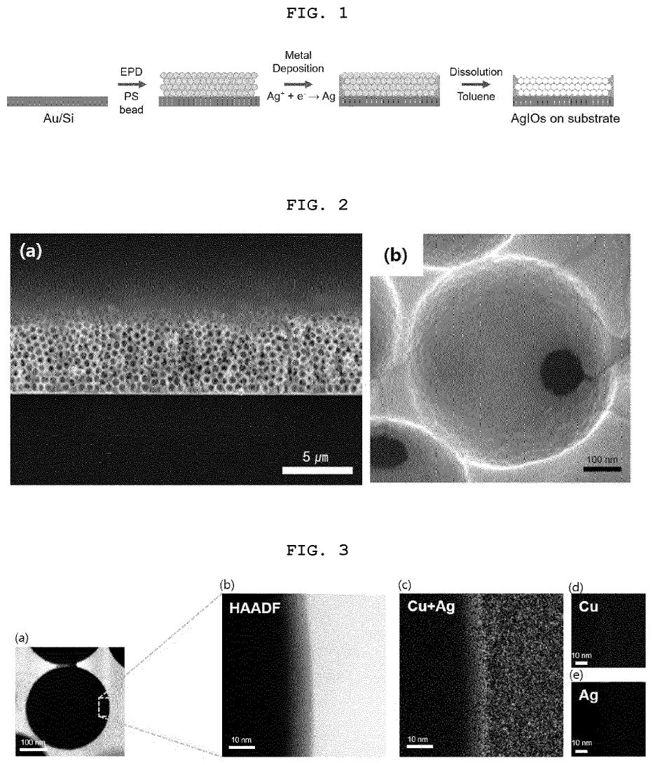

FIG. 2 is a cross-sectional SEM image (a) and a high-resolution SEM image (b) of the porous metal structure produced by the method of Example 1. It was confirmed from (a) of FIG. 2 that the porous metal framework including a plurality of pores aligned in a hexagonal structure was formed, and the pores were laminated in about 8 layers. From (b) of FIG. 2 in which the porous metal framework is enlarged, it is seen that the pores were connected to adjacent pores through small sized holes to provide a pathway through which a reactant, a reaction intermediate, and a product may move well on a catalyst surface during a carbon dioxide conversion reaction.

FIG. 3 is high-angle annular dark-field scanning transmission electron microscope (HAADF-STEM) images of the porous metal structure produced by the method of Example 1 (a, b), and energy dispersive spectrometer (EDS) mapping images thereof (c-e). It was confirmed from (b) to (e) in which the surface of the porous metal framework is enlarged that a copper thin film was electrodeposited with a very small thickness of about 7 nm or less on the surface of the porous metal framework. Pulse electrodeposition was used to control the copper thin film to be electrodeposited very thinly on the surface of the porous metal framework, thereby producing copper nanoclusters on the surface of the porous metal framework during the carbon dioxide conversion reaction. The copper nanoclusters having very high reactivity may be positioned on the surface of the porous metal framework to improve ethanol selectivity.

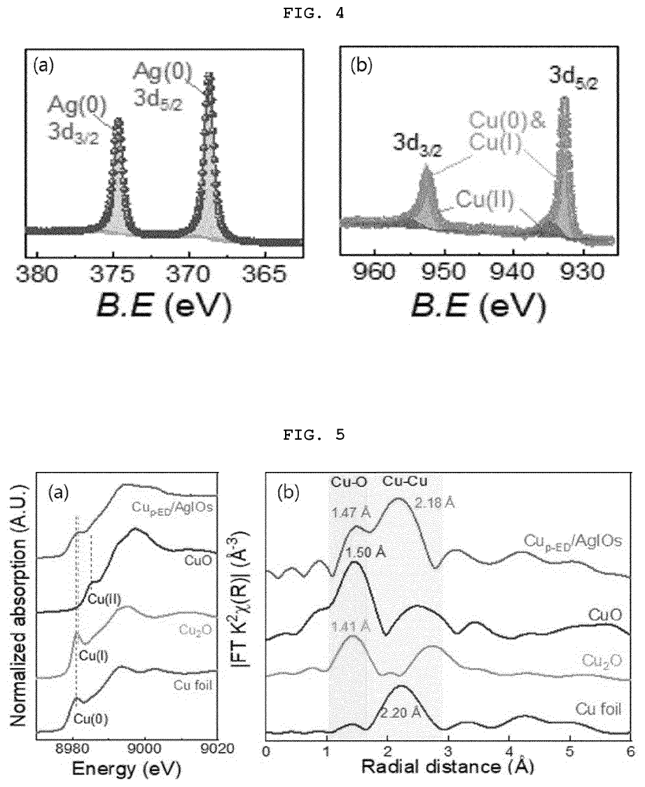

FIG. 4 is graphs illustrating XPS analysis spectra of silver (a) and copper (b) included in the porous metal structure produced by the method according to Example 1. In both silver (Ag) and copper, strong peaks were detected in 3d_{3/2} and 3d_{5/2}. In the case of copper, zero-valent (Cu⁰) and monovalent (Cu⁺) peaks were detected overlapping at about 932.6 eV, and a peak of a divalent copper (Cu²⁺) corresponding to 3p_{3/2} was detected at about 934.6 eV. In the case of silver (Ag) also, a peak for zero-valent silver (Ag⁰) was detected at about 374 eV and 369 eV.

More specifically, XANES and EXAFS analyses were performed for surface analysis of copper. FIG. 5 is graphs illustrating a k-edge X-ray absorption near edge structure (XANES) analysis spectrum (a) and an extended X-ray absorption fine structure (EXAFS) analysis spectrum (b) of the porous metal structure produced by the method according to Example 1. As a result of analyzing the X-ray absorption near edge structure (XANES), a peak was detected at about 8879 eV corresponding to transition energy of 4p_{z} at 1 s of zero-valent copper (Cu⁰) in the porous metal structure. Similarly, in the extended X-ray absorption fine structure (EXAFS) analysis spectrum of the surface of the copper thin film, a strong peak was detected at 2.18 Å corresponding to a copper-copper (Cu⁰—Cu⁰) bond length and a weak peak was detected at 1.47 Å corresponding to a copper-oxygen (Cu—O) bond length. Since a monovalent copper-oxygen bond (Cu⁺—O, Cu₂O) having a bond length of 1.41 Å and a divalent copper-oxygen bond (Cu²⁺—O, CuO) having a bond length of 1.50 Å are both included, a peak for a copper-oxygen bond was detected at 1.47 Å.

It was confirmed from the porous metal structure surface analysis in FIGS. 4 and 5 that a part of the copper thin film was naturally oxidized, but a copper-copper bond was still dominant, and since silver and copper did not exchange electronic influence with each other on the surface of the copper thin film and the porous metal framework, they were present as separate phases without forming an alloy. Therefore, during the carbon dioxide reduction, carbon dioxide may be converted into carbon monoxide with high selectivity on the surface of silver, and carbon monoxide may be easily converted into ethanol on the surface of the copper nanocluster, thereby improving selectivity to ethanol.

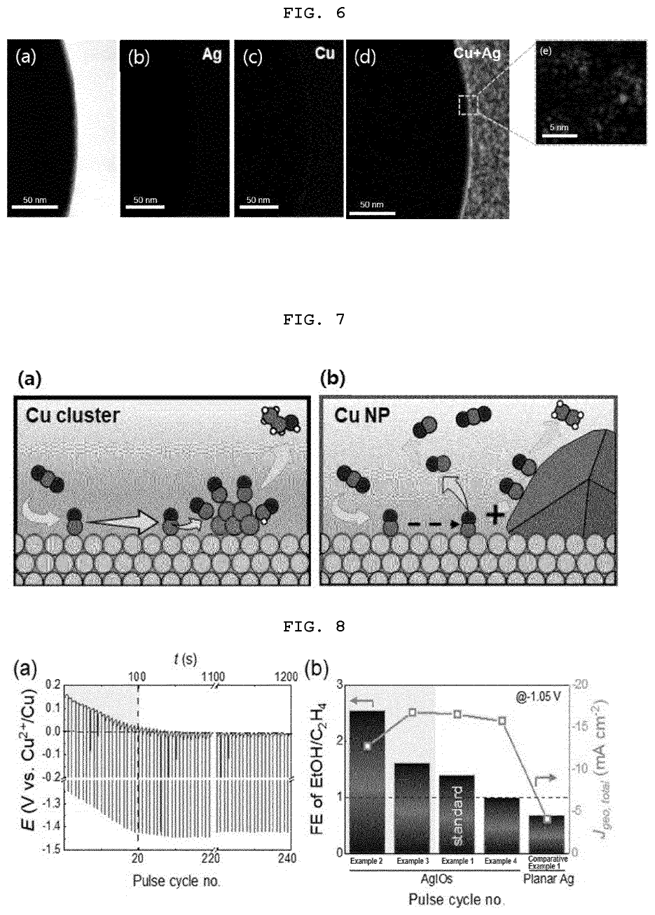

FIG. 6 is images in which the surface of the porous metal structure was analyzed after performing the carbon dioxide conversion reaction by the method of Experimental Example 1, in which (a) is a high-angle annular dark-field scanning transmission electron microscope (HAADF-STEM) image, and (b) to (e) are EDS mapping images. As seen in (c) to (e) of FIG. 6, copper nanoclusters were formed on the surface of the copper thin film, after performing the carbon dioxide conversion reaction. As in (e) of FIG. 6 in which the interface between the copper thin film and the porous metal framework is enlarged, copper particles were rearranged during the carbon dioxide conversion reaction to form nanoclusters, thereby improving a specific surface area and increasing a silver (Ag)-copper (Cu) interface length. As described above, since the copper nanoclusters has high surface area and reactivity as compared with copper nanoparticles, ethanol was able to be produced with high selectivity by dimerization of carbon monoxide on the surface of the copper nanoclusters.

FIG. 7 is schematic diagrams illustrating a carbon dioxide reduction mechanisms depending on a copper particle size included in the porous metal structure according to an exemplary embodiment. In the case of copper nanoclusters having a particle diameter of 10 nm or less, the silver (Ag) surface area of the porous metal framework was increased to produce abundant carbon monoxide from carbon dioxide on the surface of the catalyst without spatial restriction. Thus, the surface of the copper nanoclusters was occupied by carbon monoxide which may be actively converted into ethanol.

However, in the case of copper nanoparticles having a particle diameter of several hundred nanometers, since a silver (Ag) surface area was decreased to decrease a carbon monoxide production amount and an interface length to silver was decreased due to their large particle size, spatial restriction was significant when carbon monoxide diffused to the surface of the copper nanoparticle, resulting in extension of a diffusion path, and thus, ethylene was dominantly produced as compared with ethanol to reduce ethanol selectivity.

FIG. 8 is graphs in which (a) potential (V vs. Cu²⁺/Cu) and (b) Faradaic efficiency and total geometric area-based current density depending on the number of pulse cycles were measured, during pulse electrodeposition for forming the copper thin films of Examples 1 to 4 and Comparative Example 1. When the copper thin film was produced by the pulse electrodeposition, the number of pulse cycles was able to be adjusted to precisely control the thickness of the copper thin film. As the number of cycles decreased, the thickness of the copper thin film was thinned and the copper nanoclusters was able to be dominantly formed, but as the number of cycles increased, the thickness of the copper thin film was increased, so that copper nanoparticles rather than the nanoclusters were more formed.

Specifically, in Examples 2 and 3 having 20 or fewer pulse cycles, the potential was displayed as 0 V or more (yellow area), which means that the copper thin film was not completely formed, so that silver (Ag) was exposed to the surface. Therefore, since the copper nanoclusters were easily implemented, the Faradaic efficiency of ethanol was higher than that of ethylene. The Faradaic efficiency of ethanol to ethylene was 2.5 in Example 2 and 1.6 in Example 3, and thus, a large amount of ethanol as compared with ethylene was able to be produced. In Example 1 having 240 pulse cycles also, it was confirmed that the Faradaic efficiency of ethanol to ethylene was 1.4 and production of ethanol was dominant, and since the copper thin film was converted into a nanocluster form during the electrochemical conversion process of carbon dioxide, ethanol selectivity was measured to be high as compared with ethylene selectivity. However, the catalyst of Example 4 performing 1200 pulse cycles had decreased ethylene selectivity as the thickness of the copper thin film was increased.

In the case of the total geometric area-based current density (J_{geo, total}), the porous metal structure produced by the method of Examples 2 to 4 had a high total geometric area-based current density of −1.5 mA/cm², since the copper thin film was easily converted into nanoclusters by the carbon dioxide reduction reaction for a long time, but in Example 2 having the significantly decreased number of cycles, much fewer copper nanoclusters were formed and the surface area of silver (Ag) was excessively increased, so that carbon monoxide rather than ethanol became a main product to rather decrease the total geometric area-based current density. When the copper thin film was electrodeposited with 240 cycles on the surface of the flat plate-shaped silver (Ag) as in Comparative Example 1, the surface area of silver (Ag) was significantly decreased as compared with the porous metal framework to dominantly produce ethylene as compared with ethanol.

FIGS. 9 to 11 are graphs in which the current density and the Faradaic efficiency of the carbon dioxide conversion reaction depending on the applied voltage of the metal structures produced by the methods of Example 1 and Comparative Example 1 were measured. As shown in FIG. 9, Example 1 shows that as the cathodic potential was increased from −0.9 V to −1.05 V as compared with a reversible hydrogen electrode (RHE), carbon monoxide selectivity was decreased from 40% to 13%, but selectivity to ethanol was increased from 5% to 25%. In the case of methane (CH₄) and hydrogen (H₂) at −1.1 to −1.15 V, carbon monoxide was reduced due to an increase in hydrogen evolution reaction (HER) to decrease production efficiency of ethylene.

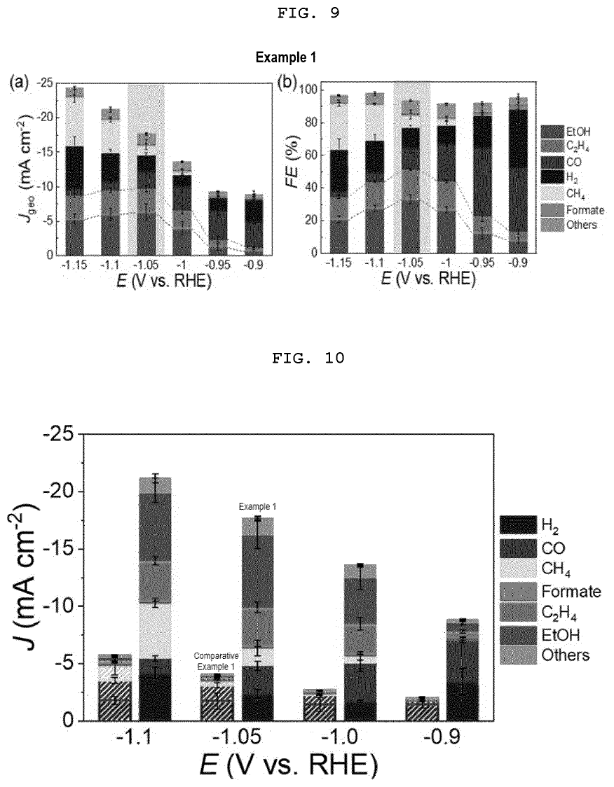

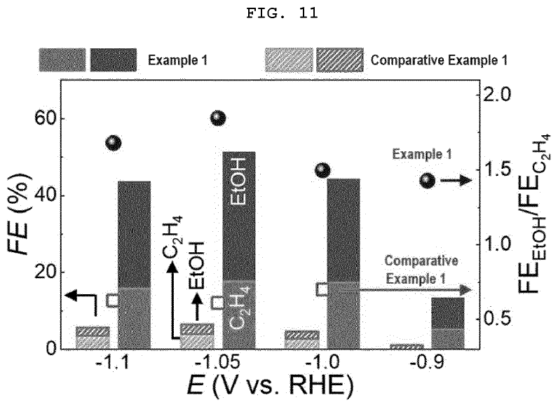

Referring to FIGS. 10 and 11, as compared with Comparative Example 1 using a flat plate-shaped silver-copper composite thin film, when the porous metal structure of Example 1 was used as a catalyst, total current density was significantly improved. In Comparative Example 1, hydrogen and carbon monoxide among the carbon dioxide reduction products were dominantly produced, but in Example 1, ethanol was produced the most. In addition, when a potential of −1.0 to −1.05 V (vs. RHE) was applied, the Faradaic efficiency of ethanol was 33.2% and high ethanol selectivity was shown as compared with ethylene, and at this time, the partial current density of ethanol was −6.31 mA/cm², and thus, about 16.4 times the ethanol selectivity and 74.7 times the partial current density as compared with Comparative Example 1 were shown. That is, since the copper nanoclusters were positioned on the surface of the porous metal framework having an inverse opal structure, the specific surface area of the catalyst was maximized, and thus, significantly high current density and Faradaic efficiency as compared with a flat plate-shaped catalyst were able to be provided, which is thus favorable.

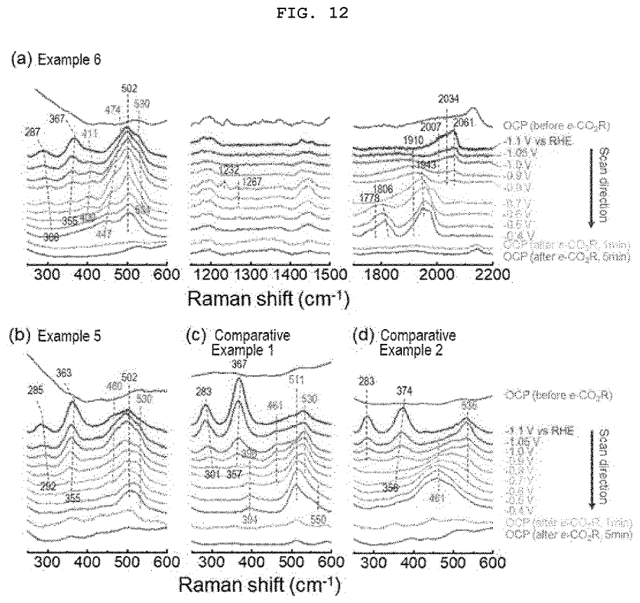

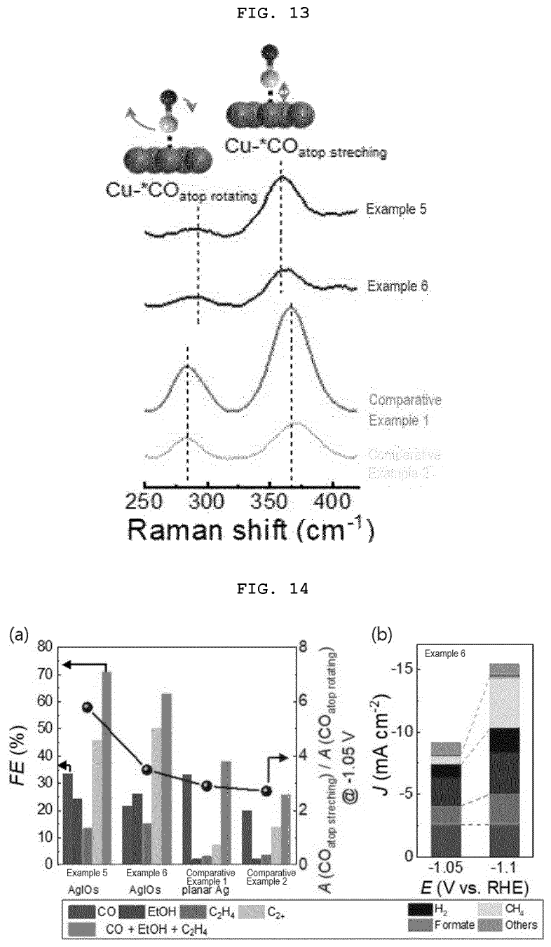

FIGS. 12 and 13 are drawings illustrating the analysis results of Operando electrochemical shell-isolated nanoparticle-enhanced Raman spectra (SHINERS) during an electrochemical reduction process of carbon dioxide using a metal structure produced by the methods of Examples 5 and 6, and Comparative Examples 1 and 2.

For SHINERS analysis, the surface of gold nanoparticles having an average particle diameter of 120 nm was evenly coated with silica having a thickness of 4 nm to produce core-shell particles, the core-shell particles were uniformly dispersed on the surface of each of the carbon dioxide conversion catalyst samples of Examples 5 and 6 and Comparative Examples 1 and 2, and then a carbon dioxide conversion process was performed to detect an analysis spectrum change during process. The core-shell particles were not involved in the oxidation and reduction reactions during carbon dioxide conversion, but in a junction between the sample and the core-shell particle, surface plasmon resonance was strengthened to strengthen the Raman signal on the surface of the sample.

The carbon dioxide conversion process was performed in a self-manufactured electrochemical cell, and an Ag/AgCl saturated with KCl was used as a reference electrode, a PT wire was used as a counter electrode, the catalysts including core-shell particles were used as a working electrode, and 36 mL of a 0.2 M KHCO₃ solution saturated with CO₂ was used as an electrolyte. A gas dispersion tube was installed under the working electrode to flow carbon dioxide gas close to the surface of the working electrode, and bubbles occurring during the carbon dioxide conversion reaction at a high potential were rapidly removed.

When the Raman signal at a potential of −0.9 V to −1.1 V where a carbon-carbon (C—C) bond occurred was analyzed, a copper-carbon monoxide (Cu-Co) stretching band was detected at 363 to 367 cm⁻¹ and a limited rotating band of a copper-carbon monoxide (Cu-Co) bond was detected at 283 to 300 cm⁻¹ on the surface of the copper thin films of Examples 5 and 6 and Comparative Examples 1 and 2. In particular, the stretching band of carbon monoxide (*CO) adsorbed on the surface of the copper thin film had better intensity than the rotating band at −1.05 V. An intensity ratio of the stretching band and the rotating band of carbon monoxide (*CO) adsorbed on the surface of the copper thin film corresponded to the amount of *CO activated to the C2 compound conversion process. It is shown that the peak intensity ratio of the stretching band to the rotating band was 5.8 in Example 5 and 3.5 in Example 6, which were high peak intensity ratios, and 2.9 in Comparative Example 1 and 2.7 in Comparative Example 2, which were low peak intensity ratios, and thus, these had lower selectivity to the C2 compound as compared with Examples 5 and 6.

When carbon monoxide (*CO) and a hydroxyl group (*OH) adsorbed on the surface of the copper thin film are excessively positioned, conversion into the C2 compound may be promoted. A C2 compound conversion pathway may be two pathways by a carbon monoxide-carbon monoxide bond (CO-CO) or by carbon monoxide-hydrocarbon bond (CO-CHₓ), and the main intermediate thereof is (H) O*CCO (H) and *COCHₓ (1302 cm⁻¹), respectively. In Examples 5 and 6, since a C—O—H stretching band of O*CCOH (1267 cm⁻¹) and a CHO peak (1232 cm⁻¹) were detected at −0.8 V, it suggested that dimerization by the carbon monoxide-carbon monoxide (CO-CO) bond was actively performed.

Since the band at 502 to 507 cm⁻¹ was observed only in Examples 5 and 6, it was confirmed that a carbon (*C) species attached to the surface of the copper thin film were involved in production of the C2 compound in the carbon dioxide conversion process using the porous metal structure as a catalyst. In addition to the *OCCOH intermediate, a wide band peak in 2700 to 2800 cm⁻¹ corresponding to the C—H stretching band of aldehyde belonging to acetaldehyde was observed at −0.7 to −1.0 V, and thus, it was confirmed that the C2 compound other than ethanol was produced. However, the yield of the C2 compound such as acetaldehyde and acetone was 2% or less which was significantly low at a potential of −1.05 V, and ethanol was dominantly formed in a yield of about 24% among the C2 compound, and thus, selectivity to ethanol was significantly improved.

Referring to FIG. 13, in the Raman spectra of Examples 5 and 6, the stretching band of a carbon monoxide-copper bond (Cu—*CO) adsorbed on the surface of the copper thin film was red shifted to 358.9 cm⁻¹ at −1.05 V, as compared with Comparative Examples 1 and 2, and the rotating band of a carbon monoxide-copper bond (Cu—*CO) was detected at 292.2 cm⁻¹, which was blue shifted as compared with Comparative Examples 1 and 2. This derived a carbon-carbon (C—C) bond between carbon monoxides (*CO) adsorbed on the surface of copper as a surface adsorption rate (coverage) of carbon monoxide was increased, thereby increasing the conversion rate to the C2 compound.

FIG. 14 is graphs of Faradaic efficiency (a) and partial current density (b) of a carbon dioxide conversion reaction including the metal structures of Examples 5 and 6 and Comparative Examples 1 and 2.

As shown in (a) of FIG. 14, Examples 5 and 6 including the porous metal structures as a catalyst provided significantly higher ethanol selectivity than Comparative Examples 1 and 2 using the flat plate-shaped thin film as a catalyst. However, as described above, though the peak intensity ratio was higher in Example 5 than in Example 6, the Faradaic efficiency to the C2 compound was higher in Example 6. Since the peak intensity ratio further included selectivity to carbon monoxide with ethanol and ethylene selectivities, peak intensity ratio of Example 5 was higher than that of Example 6, but selectivity to the C2 compound of Example 6 was measured to be substantially higher.

(b) of FIG. 14 compares current densities of ethanol and methane (CH₄) which were products at −1.05 V and −1.1 V, in the carbon dioxide conversion process including the porous metal structure produced by the method of Example 6 as a catalyst. The current densities of methane and hydrogen were increased at −1.1 V rather than −1.05 V, but the current density of ethanol was not changed at both-1.05 V and −1.1 V. The same was observed on the surface of the porous metal structure including the thick porous metal framework as in Example 1. Therefore, the ethanol active site was maintained even at −1.1 V, and the ethanol selectivity was not affected even under enhanced protonation conditions from which a carbon monoxide-hydrocarbon bond (CO—CHₓ) pathway was excluded. Therefore, it was confirmed that dimerization of carbon monoxide-carbon monoxide (CO—CO) is a main mechanism for converting carbon dioxide into ethanol in copper nanoclusters.

In summary, the content of copper which was electrodeposited on the porous metal framework was controlled using pulse electrodeposition, thereby producing a carbon dioxide conversion catalyst optimized for ethanol production. Since a copper thin film thinner than the porous metal framework was formed, copper nanoclusters having a particle diameter of several nanometers were formed on the porous metal framework to maximize a specific surface area. In addition, spatial restriction between the porous metal framework and the copper nanoclusters was minimized to increase the amount of carbon monoxide adsorbed on the surface of the copper nanoclusters to improve selectivity to ethanol. However, copper nanoparticles having a large particle size did not produce a reaction intermediate by the porous metal framework, but carbon dioxide was directly reduced on the surface of the copper nanoparticles, and thus, selectivity to ethylene (C₂H₄) was increased. In addition, as a result of SHINERS analysis, it was confirmed that ethanol was produced by carbon monoxide dimerization, not by a carbon monoxide-hydrocarbon bond (CO—CHₓ), from the O*CCOH peak. Since the current density of ethanol was constant even at a higher electrode potential, it was demonstrated that ethanol was formed by dimerization of carbon monoxide.

As a result, the porous metal structure including an inverse opal structure was used to produce a large amount of carbon monoxide on the surface of the porous metal framework having high selectivity to carbon monoxide, and the porous metal structure having significantly improved selectivity to ethanol as compared with ethylene may be provided by a large amount of bonds between carbon monoxides adsorbed on the surface of the copper nanoclusters positioned on the porous metal framework.

The porous metal structure according to the present invention may significantly improve selectivity to ethanol, when carbon dioxide is electrochemically converted.

Hereinabove, although the present invention has been described by specific matters, limited exemplary embodiments, and drawings, they have been provided only for assisting the entire understanding of the present disclosure, and the present disclosure is not limited to the exemplary embodiments, and various modifications and changes may be made by those skilled in the art to which the present disclosure pertains from the description.

Therefore, the spirit of the present invention should not be limited to the above-described exemplary embodiments, and the following claims as well as all modifications equal or equivalent to the claims are intended to fall within the scope and spirit of the invention.

What is claimed is:

1. A porous metal structure comprising:
a substrate;
a porous metal framework positioned on the substrate; and
a plurality of copper (Cu) nanoclusters positioned on a surface of the porous metal framework,
wherein the porous metal framework has an inverse opal structure including aligned pores.

2. The porous metal structure of claim 1, wherein the pores are interconnected to adjacent pores.

3. The porous metal structure of claim 1, wherein the copper (Cu) nanoclusters have an average particle diameter of 10 nm or less.

4. The porous metal structure of claim 3, wherein the plurality of copper (Cu) nanoclusters is derived from a copper (Cu)-electrodeposited thin film.

5. The porous metal structure of claim 4, wherein the copper thin film has a thickness of 1 to 10 nm.

6. The porous metal structure of claim 1, wherein the porous metal framework has a thickness of 2 to 8 μm.

7. The porous metal structure of claim 2, wherein the pores have an average particle diameter of 0.3 to 0.9 μm.

8. The porous metal structure of claim 1, wherein a metal of the porous metal framework includes one selected from the group consisting of gold (Au), silver (Ag), zinc (Zn), or a combination thereof.

9. The porous metal structure of claim 1, wherein the porous metal structure has a higher peak intensity by a copper-copper bond than a peak intensity by a copper-oxygen bond in a radial distribution function obtained by Fourier transformation of an extended X-ray absorption fine structure (EXAFS) spectrum.

10. A method for producing a porous metal structure, the method comprising:
(S10) laminating a polymer mold having an opal structure on a substrate;
(S20) electrodepositing a metal in an empty space between the polymer molds;
(S30) removing the polymer mold to form a porous metal framework having an inverse opal structure; and
(S40) pulse-electrodepositing copper (Cu) on a surface of the porous metal framework to form a copper (Cu) thin film.

11. The method for producing a porous metal structure of claim 10, wherein the polymer mold has an average particle diameter of 0.3 to 0.9 μm.

12. The method for producing a porous metal structure of claim 10, wherein in (S40), a pulse duration is 1 to 20 ms.

13. The method for producing a porous metal structure of claim 12, wherein in (S40), the number of pulse cycles is 3 to 700.

14. The method for producing a porous metal structure of claim 10, wherein (S10) is performed by electrophoretic deposition (EPD).

15. The method for producing a porous metal structure of claim 10, wherein the substrate includes a conductive material.

16. The method for producing a porous metal structure of claim 10, further comprising: after (S10), (S11) Sintering the polymer mold.

17. A carbon dioxide conversion catalyst comprising a porous metal structure including:
a substrate;
a porous metal framework positioned on the substrate; and
a plurality of copper (Cu) nanoclusters positioned on a surface of the porous metal framework,
wherein the porous metal framework has an inverse opal structure including aligned pores.

18. The carbon dioxide conversion catalyst of claim 17, wherein the catalyst has a selectivity to ethanol of 25% or more.

19. A method for producing ethanol, the method comprising:
supplying carbon dioxide to a carbon dioxide conversion system including a porous metal structure;
applying voltage to the porous metal structure to produce ethanol; and
discharging the ethanol from the carbon dioxide conversion system,
wherein the porous metal structure includes: a substrate; a porous metal framework positioned on the substrate; and a plurality of copper (Cu) nanoclusters positioned on a surface of the porous s metal framework, wherein the porous metal framework has an inverse opal structure including aligned pores.
